(19) United States
(12) Patent Application Publication
(10) Pub. No.: US 2016/0081678 A1
(43) Pub. Date: Mar. 24, 2016

(54) HINGED NEEDLE

(71) Applicant: Boston Scientific Scimed, Inc., MAPLE GROVE, MN (US)

(72) Inventors: Gary S. KAPPEL, Acton, MA (US); William BERTOLINO, Framingham, MA (US); Dennis B. HUBBARD, JR., Lancaster, MA (US); Brian GAFFNEY, Rutland, MA (US); Thomas JONES, Franklin, MA (US); Mickael A. JETTE, Shrewsbury, MA (US); Sebastian LOAIZA, Boston, MA (US); Naroun SUON, Lawrence, MA (US); Daniel FAULKNER, Cambridge, MA (US); Brian ALMEIDA, Newton, MA (US); Kimberly LAJOIE, Seattle, WA (US); Paul SMITH, Smithfield, RI (US); Eric WONG, Framingham, MA (US); Tantra S. BUDIMAN, Newton, MA (US); Justin ELFMAN, Cherry Hill, NJ (US); Vishal SHAH, Whitehall, PA (US); Steven E. WALAK, Natick, MA (US); Michael POWERS, Pepperell, MA (US); Amie PRESTON, Groton, MA (US); Mahfuza AHMED, Brookline, MA (US); Daniel R. QUINN, Littleton, MA (US)

(21) Appl. No.: 14/860,329

(22) Filed: Sep. 21, 2015

Related U.S. Application Data
(60) Provisional application No. 62/053,526, filed on Sep. 22, 2014.

(51) Int. Cl. A61B 10/06; A61B 10/02

(52) U.S. Cl. CPC A61B 10/06; A61B 10/0275; A61B 2010/0208

(57) ABSTRACT

A device for collecting a tissue sample includes a needle extending longitudinally from a proximal end to a distal end and including a channel extending therethrough, the distal end including a pair of jaws, at least a first one of the jaws being movable relative to the other between an open configuration, in which the jaws are open to receive tissue therebetween, and a closed configuration in which the jaws are drawn toward one another to collect a tissue sample therein.

### PRIORITY CLAIM

The present disclosure claims priority to U.S. Provisional Patent Application Ser. No. 62/053,526 filed Sep. 22, 2014.

### BACKGROUND

Needle biopsy procedures may be used for the diagnosis and the staging of disease. In particular, in endoscopic ultrasound-guided fine needle aspiration (EUS-FNA), the needle is advanced under ultrasound guidance so that the physician is able to visualize a position of the needle in relation to the target tissue. A distal end of the needle is then inserted into the target tissue mass to collect a sample of the tissue in a lumen thereof. Thus, EUS-FNA ensures that the correct tissue is sampled while minimizing risk to the patient. Although EUS-FNA is a highly sensitive and specific procedure, it may be difficult to acquire a suitable sample under certain clinical situations. The more cells or tissue that can be acquired, the greater the potential for a definitive diagnosis. Larger gauge needles, however, may be difficult to pass through tortuous anatomy and may acquire samples including more blood, making it more difficult to obtain a diagnosis.

### SUMMARY

The present disclosure is directed to a device for collecting a tissue sample, comprising a needle extending longitudinally from a proximal end to a distal end and including a channel extending therethrough, the distal end including a pair of jaws, at least a first one of the jaws being movable relative to the other between an open configuration, in which the jaws are open to receive tissue therebetween, and a closed configuration in which the jaws are drawn toward one another to collect a tissue sample therein.

In an embodiment, the device may further include an oversheath extending longitudinally from a proximal end to a distal end and including a lumen extending therethrough, the lumen sized and shaped to slidably receive the needle therein.

In an embodiment, a first one of the jaws may be pivotally coupled to the distal end of the needle while a second one of the jaws is fixed relative thereto.

In an embodiment, the first one of the jaws may be coupled to the needle via a hinge and movable relative thereto via a control wire extending from a distal end connected to the first one of the jaws to a proximal end extending through the channel proximally of a proximal end of the needle.

In an embodiment, the first one of the jaws may be coupled to the needle via a living hinge and is biased toward the open configuration.

In an embodiment, the jaws may be formed via a longitudinal slot extending proximally from the distal end of the needle, each jaw of the pair of jaws coupled to a remaining portion of the needle via a living hinge and is biased toward the open configuration.

In an embodiment, a longitudinal edge of the jaws may be sharpened to cut tissue as the jaws are moved to the closed configuration.

In an embodiment, the first one of the jaws may include a closed tip such that, when the jaws are in the closed configuration, a distal opening of the channel is at least partially closed.

In an embodiment, at least one of the jaws may include a plurality of barbs extending inward into the channel from an interior surface of the jaws for gripping the tissue sample received therein.

In an embodiment, a first one of the jaws may be positioned proximally of a tapered tip of the needle, a grind of the needle angled to guide tissue into the channel via the jaws when the jaws are in the open configuration.

The present disclosure is also directed to a device for collecting a tissue sample, including a needle extending longitudinally from a proximal end to a distal end and including a lumen extending therethrough and a stylet including a shaft extending longitudinally from a proximal end to a distal end and a plunger element connected to the distal end of the shaft, the plunger element positioned distally of the distal end of the lumen such that the shaft extends proximally through the lumen of the needle, the stylet movable between an open configuration, in which the plunger is separated from the distal end of the needle, and a closed configuration, in which the plunger is moved proximally toward the distal end of the needle to guide a target tissue into the lumen of the needle.

In an embodiment, the plunger may include a tapered distal tip.

In an embodiment, a proximal surface of the plunger may include a recess for receiving the target tissue as it is guided into the lumen of the needle.

In an embodiment, an edge of the proximal surface may be one of sharpened and include barbs for cutting tissue.

In an embodiment, the shaft may include a recess extending along a distal portion thereof, the recess including sharpened longitudinal edges such that rotation of the stylet, in the open configuration, about a longitudinal axis relative to the needle cores a tissue sample from the target tissue and collects the tissue sample within the recess.

The present disclosure is also directed to a method for collecting a tissue sample, comprising inserting a device to a target area within a patient body via a working channel of an endoscope, in a closed configuration in which jaws at a distal end of a needle are drawn toward one another, inserting the needle into a target tissue while moving the jaws of the needle to an open configuration in which the jaws are separated from one another to receive a portion of the target tissue therebetween, and moving the jaws to the closed configuration to collect a tissue sample within a portion of a channel of the needle defined between the jaws.

### BRIEF DESCRIPTION

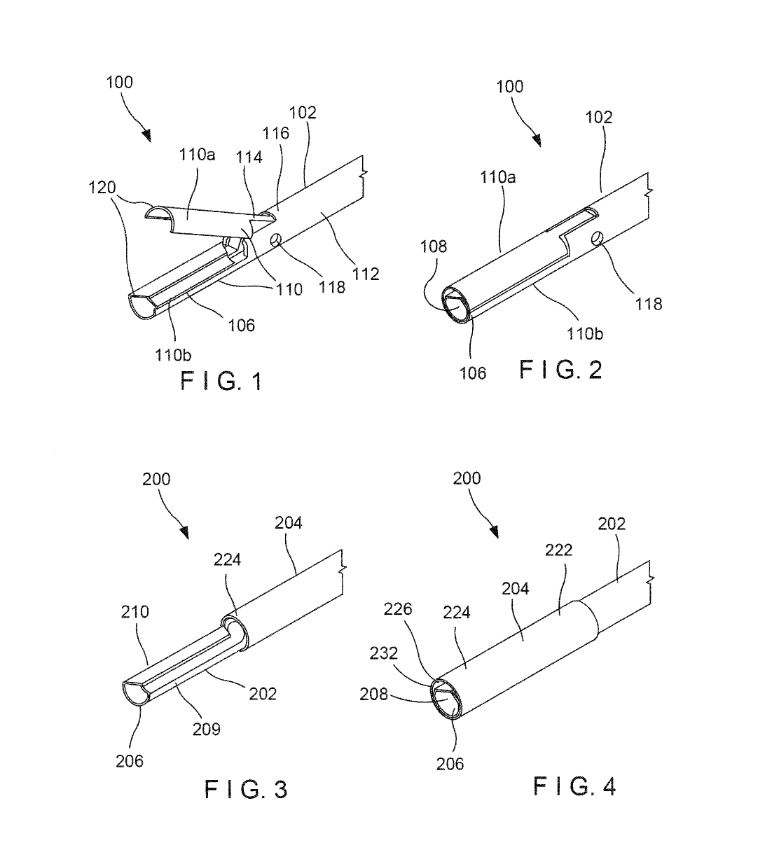

FIG. 1 illustrates a perspective view of a device according to a first exemplary embodiment of the present disclosure, in an open configuration;

FIG. 2 illustrates another perspective view of the device of FIG. 1, in a closed configuration;

FIG. 3 illustrates a perspective view of a device according to a second exemplary embodiment of the present disclosure, in an open configuration;

FIG. 4 illustrates a perspective view of the device of FIG. 2, in a closed configuration;

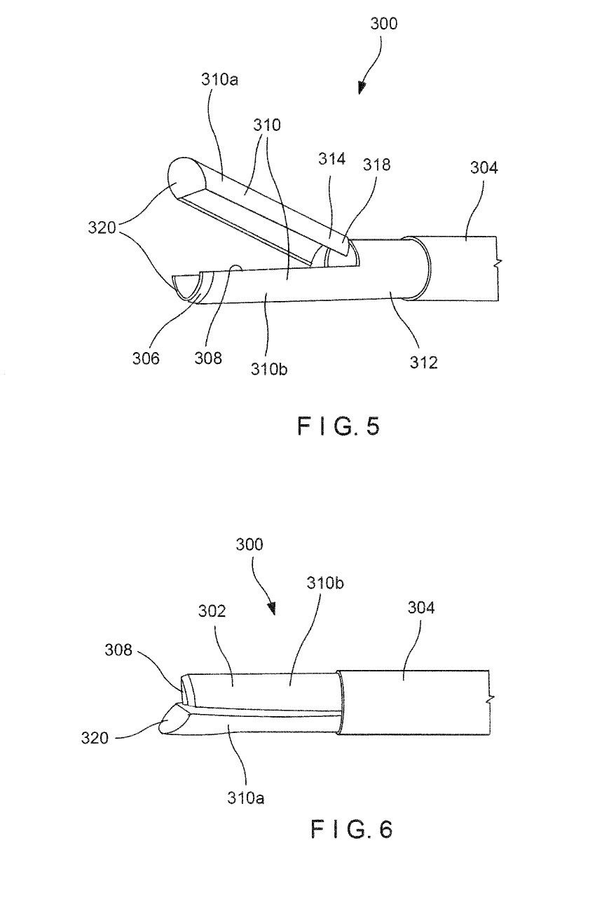

FIG. 5 illustrates a perspective view of a device according to a third exemplary embodiment of the present disclosure, in an open configuration;

FIG. 6 illustrates a perspective view of the device of FIG. 5, in a closed configuration;

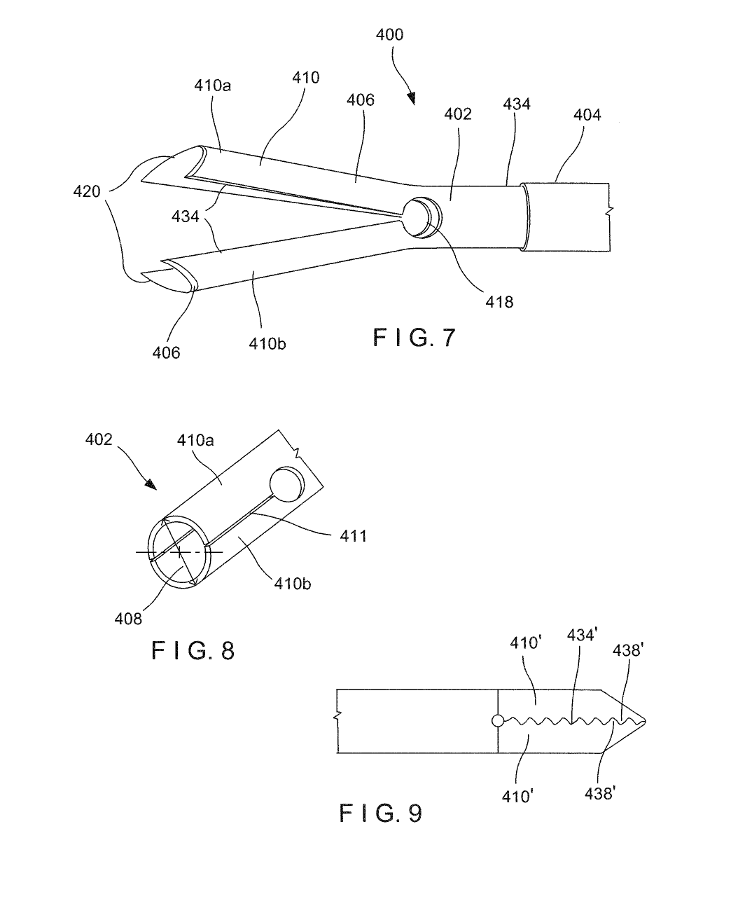

FIG. 7 illustrates a perspective view of a device according to a fourth exemplary embodiment of the present disclosure;

FIG. 8 illustrates a perspective view of a distal portion of a needle of the device of FIG. 7;

FIG. 9 illustrates a longitudinal side view of the device of FIG. 7;

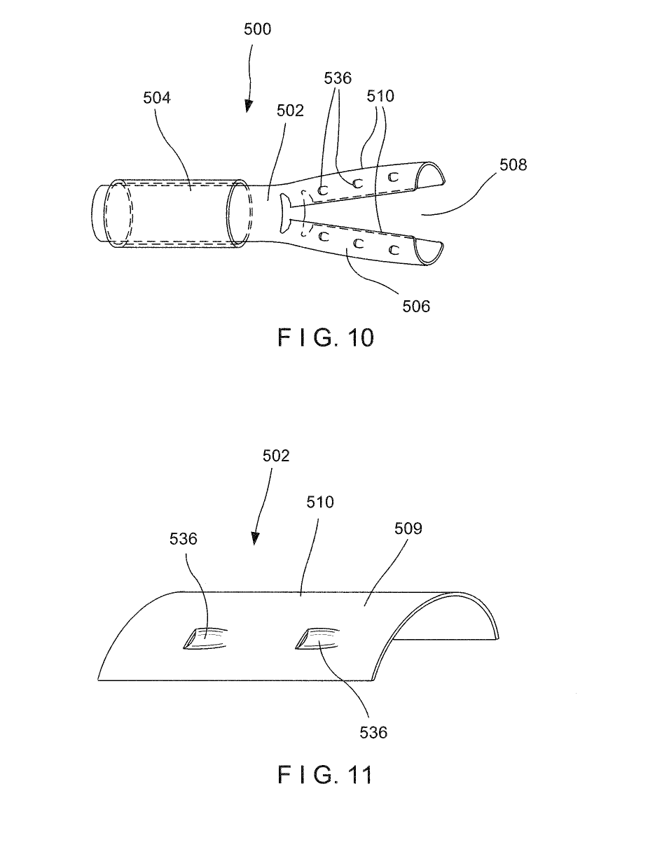

FIG. 10 illustrates a perspective view of a device according to a fifth exemplary embodiment of the present disclosure;

FIG. 11 illustrates an enlarged perspective view of a portion of a needle of the device of FIG. 10;

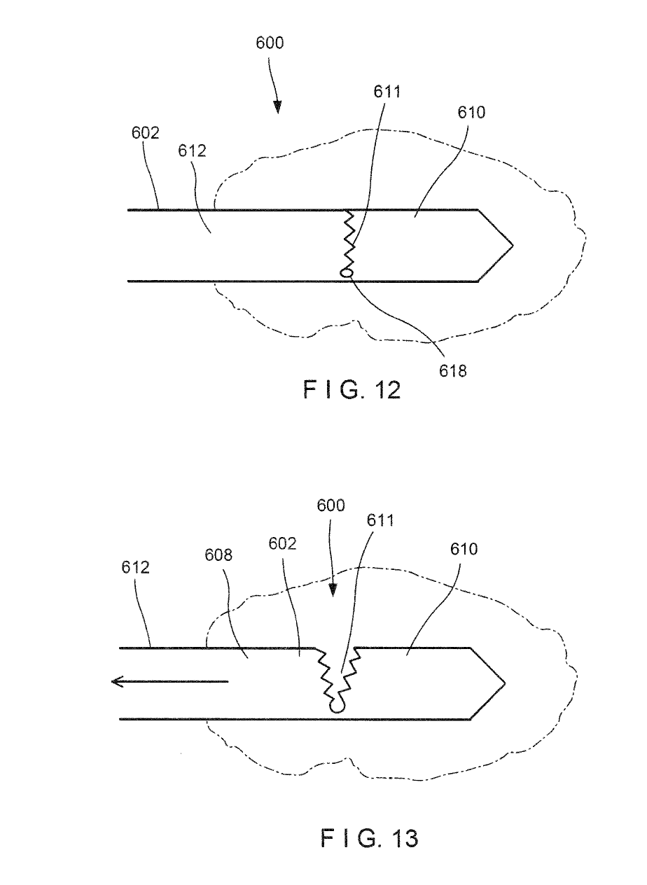

FIG. 12 illustrates a longitudinal side view of a device according to a sixth exemplary embodiment of the present disclosure, in a closed configuration;

FIG. 13 illustrates a longitudinal side view of the device of FIG. 12, in an open configuration;

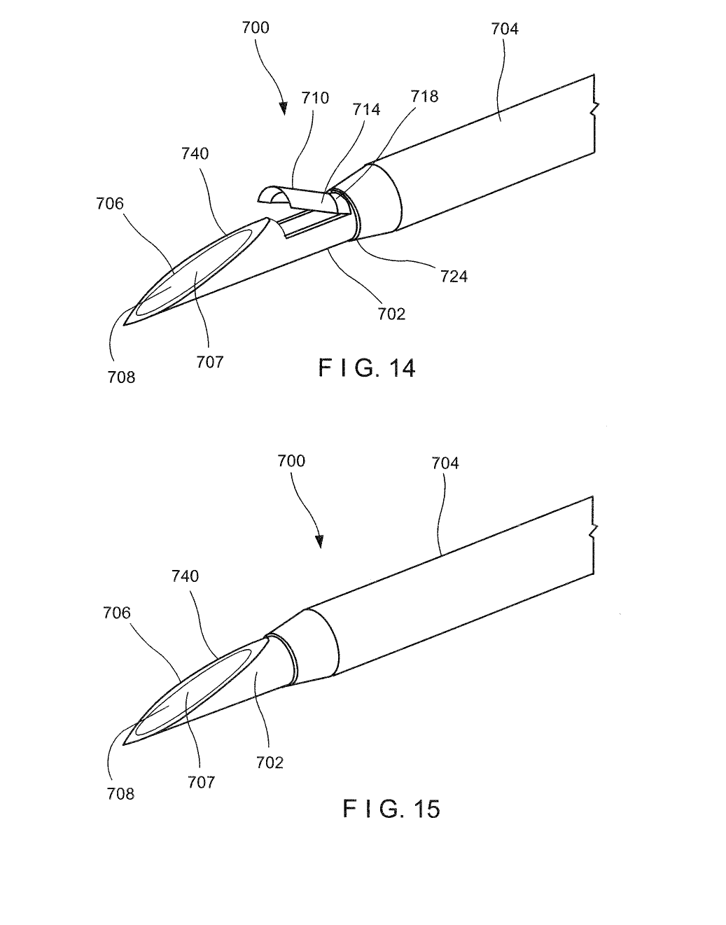

FIG. 14 illustrates a perspective view of a device according to a seventh exemplary embodiment of the present disclosure, in an open configuration;

FIG. 15 illustrates a perspective view of the device of FIG. 14, in a closed configuration;

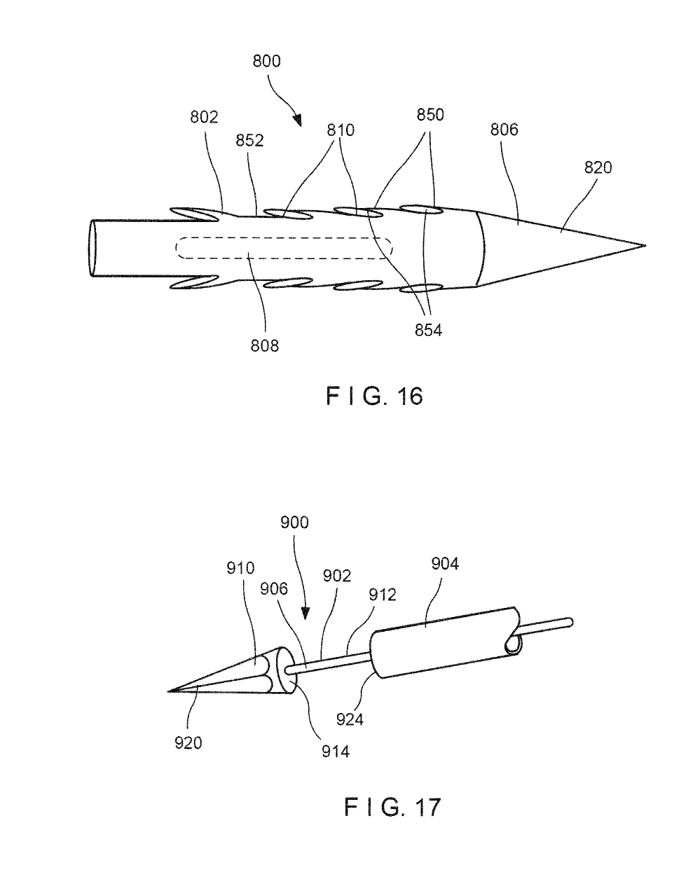

FIG. 16 illustrates a side view of a device according to an eighth exemplary embodiment of the present disclosure;

FIG. 17 illustrates a perspective view of a device according to a ninth exemplary embodiment of the present disclosure;

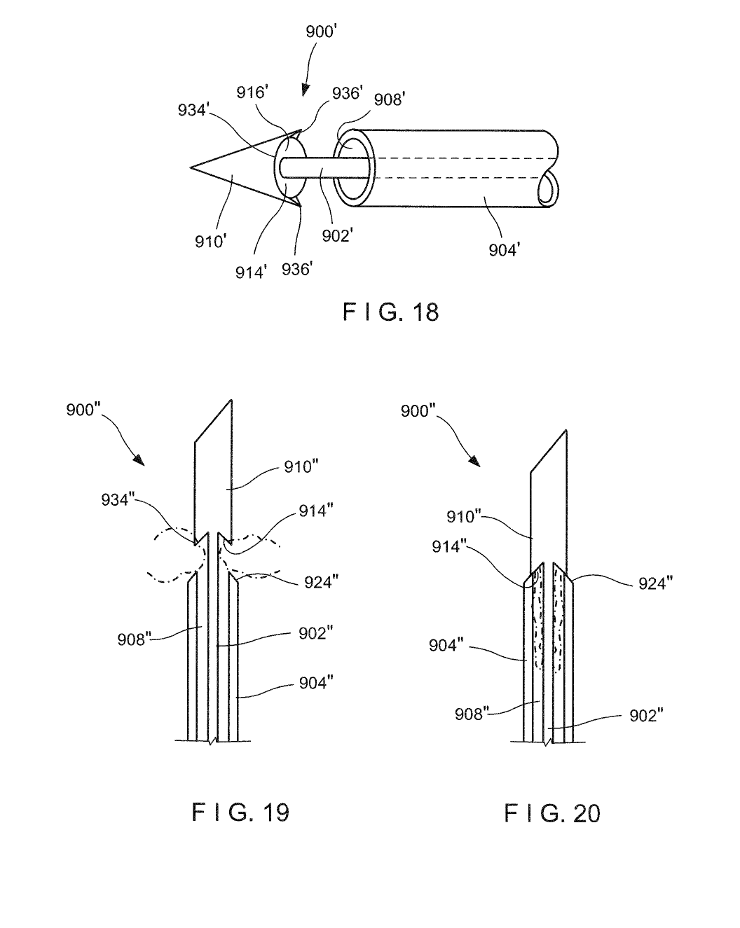

FIG. 18 illustrates a perspective view of a device according to an alternate embodiment of the present disclosure;

FIG. 19 illustrates a side view of a device according to another alternate embodiment of the present disclosure, in an open configuration;

FIG. 20 illustrates a side view of the device of FIG. 19, in a closed configuration;

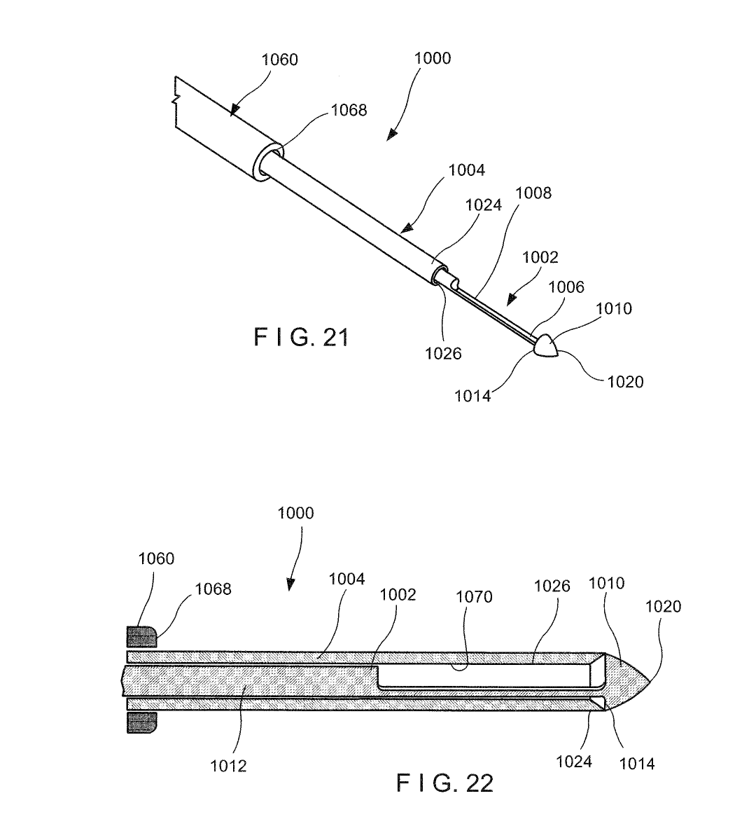

FIG. 21 illustrates a perspective view of a device according to a tenth exemplary embodiment of the present disclosure;

FIG. 22 illustrates a longitudinal cross-sectional view of the device of FIG. 21, in a closed configuration;

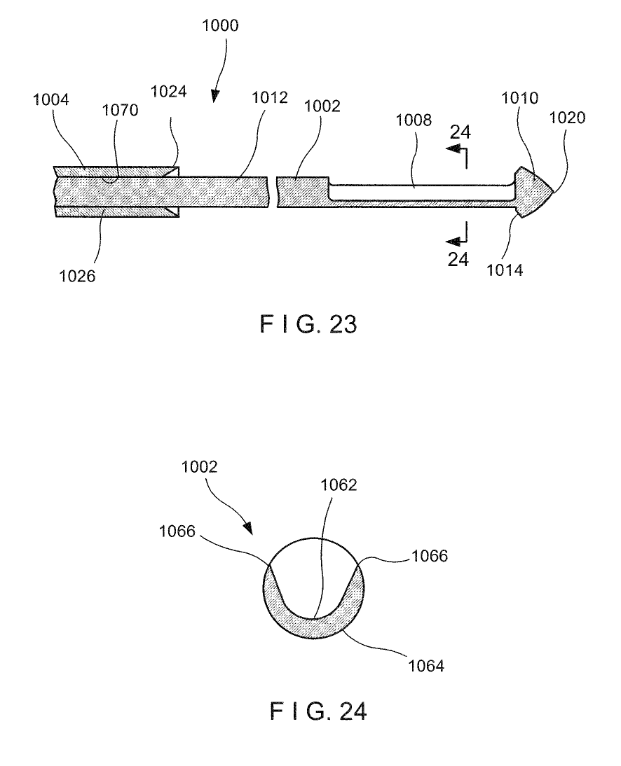

FIG. 23 illustrates a longitudinal cross-sectional view of the device of FIG. 21, in an open configuration; and

FIG. 24 illustrates a lateral cross-sectional view of a stylet of the device of FIG. 21, along a line A-A shown in FIG. 22.

### DETAILED DESCRIPTION

The present disclosure may be further understood with reference to the following description and the appended drawings, wherein like elements are referred to with the same reference numerals. The present disclosure relates to devices for obtaining tissue samples and, in particular, to EUS-FNA devices. Exemplary embodiments of the present disclosure describe devices comprising a needle including jaws at a distal end thereof movable between an open configuration for receiving a tissue sample therebetween and a closed configuration for collecting the tissue sample. The movable jaws increase a tissue acquisition area beyond a diameter of the needle so that larger core tissue samples may be obtained. It should be noted that the terms “proximal” and “distal” as used herein, are intended to refer to a direction toward (proximal) and away from (distal) a user of the device.

As shown in FIGS. 1-2, a device 100 according to a first exemplary embodiment of the disclosure comprises a needle 102 sized and shaped for insertion through an insertion device (e.g., a working channel of a flexible endoscope) to a target tissue site within a patient's body. The needle 102 extends along a longitudinal axis from a proximal end (not shown) to a distal end 106 and includes a channel 108 extending therethrough. The needle 102 further includes a pair of jaws 110 at the distal end 106. The jaws 110 are movable between an open configuration, in which the jaws 110 are separated from one another to receive tissue and a closed configuration, in which the jaws 110 are moved toward one another to collect tissue gripped therebetween. The device 100 further comprises a handle assembly (not shown) coupled to the proximal end of the needle 102 which remains outside the body accessible to a user. The handle according to this embodiment includes an actuator for moving the jaws 110 between the open and closed configurations as would be understood by those skilled in the art.

The jaws 110 are formed so that they substantially continue an outer surface of the needle 102. Thus, when in the closed configuration, an interior of the jaws 110 defines a distal portion of the channel 108. In one embodiment, a first one of the jaws 110a is pivotally coupled to a remaining portion 112 of the needle 102 such that the first jaw 110a is movable relative to a second one of the jaws 110b, which is not movable relative to the remaining portion 112 of the needle 102. In particular, a proximal end 114 of the first jaw 110a is coupled to a distal end 116 of the remaining portion 112 of the needle 102 via a hinge 118 about which the first jaw 110a pivots. Thus, in the open configuration, the first jaw 110a is pivoted about the hinge 118 away from a longitudinal axis of the device 100 such that distal ends 120 of the jaws 110 are separated from one another to receive tissue therebetween. In the closed configuration, the first jaw 110a is pivoted toward the longitudinal axis so that the pair of jaws 110 together define the distal portion of the channel 108. A distal end 104 of the channel 108 may be open such that the jaws 110 may be inserted into target tissue in the open configuration so that a portion of tissue is received within a portion of the channel 108 defined by the second jaw 110a. The jaws 110 are then moved to the closed configuration such that a larger tissue sample may be collected in the channel 108, between the jaws 110. Although the exemplary embodiment shows and describes a hinge 118, in another embodiment, the first jaw 110a may be assembled with the remaining portion of the needle 102 via a snap mechanism. For example, protrusions along a portion of the first jaw 110a may be received within a recess within a portion of the needle 102 so long as the snap mechanism permits the first jaw 110a to be pivoted thereabout. In addition, although the exemplary embodiments show and describe two jaws 110a, 110b, the needle 102 may include any number of jaws 110 so long as the jaws 110 are movable toward and away from one another, between closed and open configurations.

The jaws 110 may be moved between the open and the closed configurations via, for example, a control wire. A distal end of the control wire may be coupled to an interior surface of the first jaw 110a and/or a portion of the hinge 114 such that the wire extends proximally therefrom through the channel 108 to a proximal end which extends proximally of the proximal end of the needle 102 to be accessible to a user of the device. Thus, pushing the control wire distally relative the needle 102 moves the jaws 110 to the open configuration while drawing the control wire proximally relative to the needle 102 moves the jaws 110 to the closed configuration. In a further embodiment, the jaws 110 may be biased toward either of the open and closed configurations via a spring element coupled, for example, to the control wire or to an actuator. For example, where the spring element biases the jaws 110 toward the open configuration, an actuator on the handle assembly may fix the control wire relative to the needle 102 such that the needle 102 may be inserted through the working channel of the endoscope in the closed configuration. Once the needle 102 has reached the target tissue site, however, the actuator may be released to permit the jaws 110 to revert under their bias to the open configuration. Upon receipt of a portion of the target tissue between the jaws 110, the control wire may be drawn distally (via the actuator) to move the jaws 110 to the closed configuration, collecting the tissue sample in the channel 108 defined thereby. In another embodiment, the device 100 may further comprise an oversheath movable over the needle 102 to move the needle 102 between the open and closed configurations.

The jaws 110 may also include spikes, serrations, teeth, etc., along interior surfaces thereof to aid in the capture and collection of the tissue sample therein. In particular, the first jaw 110a, which pivots about the hinge 114, may include a spike, serration, teeth, etc. to hold tissue as the first jaw 110a is moved relative to the second jaw 110b. Both jaws 110, however, may include one or more of such tissue grabbing features such as spikes, serrations, teeth, micro-pattern, a tissue-holding sticky or adhesive material, etc.

Although the exemplary embodiment shows and describes the proximal end 114 of the first jaw 110a coupled to the remaining portion 112 of the needle 102, in another embodiment, the distal end 120 of the first jaw 110a may be pivotally coupled to the needle 102 so that tissue may be collected in the channel 108 between the proximal ends 114 of the jaws. In yet another embodiment, rather than being pivotally coupled to the remaining portion 112 of the needle 102, the first jaw 110a may be coupled thereto via a four bar linkage such that the first jaw 110a remains parallel to the second jaw 110b as the jaws 110 are moved between the open and closed configurations. The first jaw 110a may be coupled to the remaining portion 112 of the needle 102 using any of a variety of known linkage type mechanisms allowing the jaws 110 to be moved relative to one another to capture and trap tissue therebetween.

According to an exemplary method using the device 100, the device 100 is maintained in the closed configuration as it is inserted to a target tissue site within a patient's body via an insertion device (e.g., through the working channel of an endoscope). Upon reaching the target tissue site, the jaws 110 are moved to the open configuration and inserted into the target tissue. The jaws 110 are then moved to the closed configuration to capture and collect tissue between the jaws 110, within the channel 108 defined thereby. As described above, the opening and closing of the jaws 110 permits a larger sample to be collected in the channel 108 as compared to a sample that could be harvested using a needle of the same size without such movable jaws.

As shown in FIGS. 3-4, a device 200 according to a second exemplary embodiment of the present disclosure comprises a needle 202 and an oversheath 204 longitudinally slidable thereover between an open configuration, in which a window 210 at a distal end of the needle 202 is exposed, and a closed configuration in which the window 210 is covered by the oversheath 204. In particular, the window 210 extends proximally from a distal end 206 such that a distal portion of the channel 208 is open to an exterior of the needle 202. Thus, the device 200 may be inserted into a target tissue, in the open configuration, such that a tissue sample is received within the open distal portion of the channel 208. The oversheath 204 is then moved distally over the needle 202 to the closed configuration to capture the tissue within the channel 208 via an interior surface 232 of the oversheath 204.

The needle 202 extends longitudinally from a proximal end to the distal end 206 and includes the channel 208 extending longitudinally therethrough. The window 210 extends proximally from distal end 206, laterally through a wall 209 defining the needle 202 such that the distal portion of the channel 208 is open to the exterior of the needle. In one exemplary embodiment, the window 210 may extend approximately halfway about a circumference of the needle 202 along a length corresponding to a desired length of a tissue sample to be collected. The window 210, however, may have any of a variety of shapes and sizes so long as the window 210 exposes the distal portion of the channel 208 to an exterior of the needle 202.

The oversheath 204 extends longitudinally from a proximal end 222 to a distal end 224 and includes a lumen 226 extending therethrough, the lumen 226 is sized and shaped to slidably receive the needle 202 therein. The oversheath 204 may be moved longitudinally relative to the needle 202 via, for example, a control wire. For example, a distal end of the control wire may be coupled to a portion of the oversheath 204 while a proximal end is accessible to a user of the device 200 via, for example, a handle assembly coupled to a proximal end of the device 200. In the open configuration, the distal end 224 of the oversheath is proximal of a proximal end 228 of the window 210 so that the window 210 is exposed to permit tissue to be passed therethrough into the distal portion of the channel 208. In the closed configuration, the oversheath 204 is moved distally relative to the needle 202 so that the oversheath 204 substantially covers the window 210 to capture a tissue sample within the channel 208 between an interior surface 230 of the needle 202 at the distal end 206 and the interior surface 232 of the oversheath 204.

The device 200 is inserted to a target tissue site in the closed configuration through, for example, a working channel of an endoscope in the closed configuration. Once the device 200 reaches the target tissue site, however, the device 200 is moved to the open configuration by drawing the oversheath 204 proximally relative to the needle 202 and the device 200 is inserted into the target tissue in the open configuration. Insertion of the device 200 into the target tissue causes tissue to be received within the distal portion of the channel 208 via the window 210. The oversheath 204 is then moved distally over the needle 202 into the closed configuration to capture the tissue sample within the channel 208 and sever it from surrounding tissue.

As shown in FIGS. 5-6, a device 300 according to a third exemplary embodiment of the disclosure is substantially similar to the devices 100, 200 described above except as noted below. The device 300 comprises a needle 302 and an oversheath 304. The needle 302 is substantially similar to the needle 102, describe above in regard to the device 100, comprising a pair of jaws 310 at a distal end 306 thereof and the oversheath 304 is substantially similar to the oversheath 204 described above. The oversheath 304 is longitudinally movable over the needle 302 to move the jaws 310 of the needle 302 between an open configuration, in which distal ends 320 thereof are separated from one another, and a closed configuration, in which the distal ends 320 of the jaws 310 are moved toward one another. The jaws 310 in this embodiment are biased toward the open configuration such that, as the oversheath 304 is moved distally over the jaws 310, contact with the oversheath 302 forces the jaws into the closed configuration.

Similarly to the needle 102, in this embodiment only one of the jaws 310a of the needle 302 is pivotally coupled to a remaining portion 312 of the needle 302 while a second one of the jaws 310b is fixed relative to the remaining portion 312. Those skilled in the art will understand, however, that for any of the embodiments including jaws, either or both of the jaws may be movable between the open and closed configurations. A proximal end 314 of the first jaw 310a is connected to the remaining portion 312 of the needle via, for example, a living hinge 318 which biases the jaws 310 toward the open configuration. In the open configuration, the first jaw 310a is pivoted away from a longitudinal axis of the device 300 so that distal ends 320 of the jaws 310 are separated from one another. In the closed configuration, the oversheath 304 is moved distally over the needle 302 to extend over the jaws 310 so that the jaws 310 are constrained via an interior surface of the oversheath 304.

The first jaw 310a in this embodiment is closed at a distal end 320 thereof such that when the jaws 310 are moved to the closed configuration, a tissue sample is captured in the channel 308 between the jaws 310 and prevented from being inadvertently released therefrom via a distal opening. The closed distal end 320 of may also be tapered such that, in the closed configuration, the tapered end may be used to navigate a sample region therewith. As will be understood by those of skill in the art, the oversheath 304 is not required to extend over the entire length of the jaws 310 to constrain the jaws 310 in the closed configuration.

Except as noted below, the device 300 is used in a manner substantially similar to the device 100, 200 described above. In particular, the device 300 is inserted through an insertion device to a target tissue site, in the closed configuration. Upon reaching the target tissue site, the needle 302 may be moved distally relative to the oversheath 304 so that the jaws 310 are permitted to revert to the biased open configuration as the needle 302 is inserted into the target tissue. Once tissue has been received between the jaws 310, the oversheath 304 are moved distally over the needle 302 to close the jaws 310, capturing a tissue sample in the channel 308 between the jaws 310.

As shown in FIGS. 7-8, a device 400 according to a fourth exemplary embodiment of the present disclosure is substantially similar to the device 300 described above (except as noted), comprising a needle 402 including jaws 410 at distal end 406 thereof. An oversheath 404 is longitudinally movable over the needle 402 to move the jaws 410 between an open configuration in which distal ends 420 of the jaws 410 are separated from one another to receive tissue, and a closed configuration in which distal ends 420 of the jaws 410 are drawn toward one another to capture a tissue sample in a channel 408 defined by the jaws 410. The needle 402 of this embodiment is substantially similar to the needle 302. The jaws 410 of the needle 402, however, are both movable relative to a longitudinal axis of the needle 402. Each of the jaws 410 is connected to a remaining portion 412 of the needle 402 via a living hinge 418. In particular, the jaws 410 may be formed via a longitudinal slot 411 extending proximally from the distal end 406 to define the two jaws 410. The jaws 410 are biased toward the open configuration so that, when the oversheath 404 is moved distally thereover, the jaws 410 are constrained to the closed configuration as an interior surface 432 of the oversheath contacts the jaws 410 forcing them radially inward to the closed configuration.

The distal ends 420 of each of the jaws 410 may be beveled to facilitate insertion thereof into the target tissue. Suction may also be applied through the channel 408 to draw tissue between the jaws 410. The beveled distal ends 420 and longitudinal edges 434 of the jaws 410 may also be sharpened to facilitate cutting the tissue sample away from the surrounding target tissue as the jaws 410 are drawn toward one another into the closed configuration. In another embodiment, as shown in FIG. 9, longitudinal edges 434′ of jaws 410′ may include teeth 438′ or serrations to aid in cutting of the tissue sample from a surrounding target tissue. In addition a distal end 424 of the oversheath 404 may also include a sharpened edge so that the target tissue is cut as the oversheath 404 is moved distally over the jaws 410. The oversheath 404 may also be rotated bout the longitudinal axis as it is advanced distally over the jaws 410 to aid in cutting of the tissue. It will be understood by those of skill in the art that the device 400 may be used in a manner substantially similar to the device 300.

As shown in FIGS. 10-11, a device 500 according to a fifth exemplary embodiment of the present disclosure is, except as noted, substantially similar to the device 400 described above. The device 500 comprises a needle 502 including jaws 510 at a distal end 506 thereof and an oversheath 504 longitudinally movable relative thereto between an open configuration, in which the jaws 510 are separated from one another, and a closed configuration, in which the jaws 510 are drawn toward one another. As the jaws 510 are moved to the closed configuration, a sample of the tissue received between the jaws 510 is captured in a channel 508 between the jaws 510. The jaws 510 of the needle 502, further include barbs 536 extending into the channel 508 defined by the jaws 510 for gripping tissue received therein. The barbs 536 may be formed as tabs cut through a wall 509 of the needle 502 and bent inward toward a longitudinal axis of the needle 502 to hook or grip tissue received therein.

As shown in FIGS. 12-13, a device 600 according to a sixth exemplary embodiment of the present disclosure is, except as noted below, substantially similar to the device 100 comprising a needle 602 including a distal jaw 610 movable between an open configuration and a closed configuration to capture a tissue within a channel 608 defined thereby. Rather than being formed via a longitudinal slot extending from proximally from a distal end of the needle, however, the jaw 610 is formed via a slot 611 extending laterally through the needle 602 such that the distal jaw 610 is movable relative to a proximal portion 612 of the needle 602. The distal jaw 610 is coupled to the proximal portion 612 via a hinge 618 about which the distal jaw 610 pivots relative to the proximal portion 612, between the open configuration, in which the distal jaw 610 is pivoted away from the proximal portion 612 to be separated therefrom, and the closed configuration, in which the jaw 610 is pivoted toward the proximal portion 612 to close a space therebetween.

Suction is applied through the channel 608 to draw tissue into a space between the distal jaw 610 and the proximal portion, in the open configuration. Once tissue has been received in the channel 608 between the distal jaw 610 and the proximal portion 612, the jaw 610 is moved to the closed configuration to capture a tissue sample in the channel 608. The edges defining the lateral slot 611 may be sharpened and/or include teeth or serrations for cutting the tissue sample from the surrounding tissue. The jaw 610 may be moved between the open and closed configurations via, for example, a control wire substantially similar to the control wire described above in regard to the device 100.

As shown in FIGS. 14-15, a device 700 according to a seventh exemplary embodiment of the present disclosure is, except as noted below, substantially similar to the device 200 and comprises a needle 702 including a jaw 710 pivotally coupled thereto for movement between an open configuration and a closed configuration via an oversheath 704. The jaw 710, however, is positioned proximally of a tapered tip 706 of the needle 702 and is formed by cutting a flap through a wall of the needle 702 proximal of the tapered tip 702 such that a proximal end 714 of the flap remains connected to the needle 702. The jaw 710 may, for example, be heat set in the open configuration with the jaw 710 bent away from the longitudinal axis of the needle 702 such that the jaw 710 is biased toward the open configuration. When the jaw 710 is in the open configuration, a channel 708 extending longitudinally through the needle 702 is open to an exterior of the needle 702 via both the open jaw 710 and a distal opening 707 of the channel 708. The tapered tip 706 includes a grind 740 angled so that, when the needle 702 is inserted into a target tissue, tissue is guided into the open jaw 710. Thus, a tissue sample may be received in the channel 708 of the needle 702 via both the distal opening 707 and the open jaw 710. Once the tissue sample has been received in the channel 708, the oversheath 704 is moved distally over the needle 702 to move the jaw 710 to the closed configuration in which the jaw 710 is constrained via an interior surface of the oversheath 704. Although the exemplary embodiment shows and describes the jaw 710 as being coupled to the needle 702 at the proximal end 714, in an alternate embodiment, the jaw 710 may be coupled to the needle 702 at a distal end thereof. The jaw 710 may be moved between the open and closed configurations via a spring and the oversheath 704, which may be moved thereover.

In use, the device 700 is inserted to a target tissue site in a patient's body via an insertion device such as through a working channel of an endoscope. The device 700 is inserted through the working channel in an insertion configuration with the oversheath 704 extending distally over the tapered tip 706 to prevent the tapered tip 706 from damaging the endoscope as it is inserted therethrough. Upon reaching the target tissue site, however, the oversheath 704 is drawn slightly proximally relative to the needle 702 so that the tapered tip 706 is exposed while the jaw 710 is still covered by the oversheath 704 and is in the closed configuration. In this position, the device 700 is advanced so that the tapered tip 706 pierces the tissue. Needle 702 is then moved distally relative to the oversheath 704 permitting the jaw 710 to move to the open configuration. As described above, the grind 740 along the tapered tip 706 is specifically selected so that tissue is guided into channel via the open jaw 710 as the needle 702 is moved from the closed configuration to the open configuration. Thus, tissue is received in the channel 708 via both the distal opening 707 of the channel 708 and the open jaw 710. Once the tissue has been collected, as desired, the oversheath 704 is moved distally over the jaw 710 to return the jaw 710 to the closed configuration, trapping the collected tissue in the channel 708. Similarly to the oversheaths described above, the oversheath 704 may include a sharpened distal edge 724 which severs the tissue sample from surrounding tissue as it is moved distally over the jaw 710 to the closed configuration.

As shown in FIG. 16, a device 800 according to an eighth exemplary embodiment of the present disclosure comprises a needle 802 extending longitudinally from a proximal end to a distal end 806 and including a channel 808 extending therethrough. A distal portion of the needle 802 includes a plurality of openings 810 extending laterally through a wall thereof and a plurality of extending members 850 extending from an exterior surface 852. The openings 810 extend through the wall in communication with the channel 808. Each of the extending members 850 extends from the exterior surface 852 at an angle relative to and adjacent to a corresponding one of the openings 850. For example, each extending member 850 extends at an angle pointing toward the proximal end of the needle 802, immediately distal to a corresponding one of the openings 810 so that, when the needle 802 is inserted into target tissue and then drawn proximally relative thereto, tissue is guided via angled surfaces 854 of the extending members 850 through the openings 810 into the channel 808. In another embodiment, each extending member 850 extends at an angle toward the distal end 806 immediately proximal to a corresponding one of the openings 810 such that inserting the needle 802 distally into the target tissue causes tissue to be guided along the angled surfaces 854 through the openings 810 and into the channel 808.

The distal end 806 of the needle 802 includes a closed, break away tip 820 for removing the tissue sample collected within the channel 808. For example, once the tip 820 has been broken off from a remaining portion 812 of the needle 802, a pusher may be inserted into the channel 808 to push the tissue sample therein through the channel 808 and out of the distal end 806. Although the exemplary embodiment only shows and describes a needle 802, the device 800 may further comprise an oversheath longitudinally movable over the needle 802 such that, when the device 800 is inserted through, for example, a working channel of an endoscope, the oversheath may extend over the distal portion of the needle 802 to cover the extending members 850 and openings 810, preventing the extending members 850 from damaging the working channel. Once the device 800 has reached the target tissue site, however, the needle 802 is moved distally relative to the oversheath so that the needle 802 may be inserted into the target tissue to collect a tissue sample. Upon collection of the tissue sample, the oversheath is moved distally over the needle 802 to cover the plurality of openings 810 and extending members 850 to facilitate removal of the device 800 from the patient's body.

As shown in FIG. 17, a device 900 according to a ninth exemplary embodiment of the present disclosure comprises a stylet 902 and a needle 904. The stylet 902 includes a shaft portion 912 extending longitudinally from a proximal end to a distal end 906. The distal end 906 of the stylet 902 includes a plunger element 910 having a larger cross-sectional area than a remaining portion of the stylet 902. The plunger element 910 includes a tapered distal tip 920 for piercing tissue into which it is inserted. The needle 904 extends longitudinally from a proximal end to a distal end 924 and includes a lumen 926 extending therethrough. The plunger element 910 is positioned distally of the distal end 924 of the needle 904 while the shaft portion 912 extends proximally through the lumen 926. The stylet 902 is movable between an open position, in which the plunger element 910 is spaced from the distal end 924 of the needle 904, and a closed position, in which a proximal end 914 of the plunger element 910 is drawn toward the distal end 924 of the needle 904.

In use, the device 900 is inserted into target tissue in the closed position. Once inserted into the target tissue, the stylet 902 is moved distally relative to the needle 904 to the open position. The stylet 902 is then again moved proximally relative to the needle 904 toward the closed configuration such that tissue is guided into the lumen 926 via the proximal surface 914 of the plunger 910. Alternatively, once inserted into the target tissue, the needle 940 may be moved distally relative to the stylet 902 to the open position. The needle 904 is then again moved proximally relative to the stylet 902 toward the closed configuration such that tissue is guided into the lumen 926 via the distal end of the needle 924.

In an alternate embodiment, as shown in FIG. 18, a device 900′ comprises a stylet 902′ including a plunger 910′ having a recess 916′ at a proximal surface 914′ thereof for increasing an area in which tissue may be collected between the proximal surface 914′ of the plunger 910′ and into a channel 908′ of a needle 904′. In one embodiment, the recess 916′ is configured to form a concave proximal surface 914′. Edges 934′ of the proximal surface 914′ extending about a periphery thereof may be sharpened and/or include barbs 936′ to facilitate cutting of the tissue as the stylet 902′ is drawn proximally relative to the needle 904′.

According to another alternate embodiment, as shown in FIGS. 19-20, a device 900″ is, except as noted, substantially similar to the device 900′ described above, comprising a stylet 902″ including a plunger 910″ and a needle 904.″ A proximal surface 914″ of the plunger 910″, is angled such that an edge 934″ of the proximal surface 914″ extends proximally of a remaining portion of the proximal surface 914.″ The edge 934″ thus grips and cuts tissue as the stylet 902″ is moved proximally relative to the needle 904.″ A distal end 924″ of the needle 904″ also includes a sharpened edge to cut tissue moved theretoward by the plunger 910″, collecting and capturing a tissue sample in a channel 908″ thereof.

As shown in FIGS. 21-24, a device 1000 according to another exemplary embodiment of the present disclosure may be substantially similar to the device 900 described above, comprising a stylet 1002 slidably received within a needle 1004. The device 1000 further comprises a sheath 1060 in which the stylet 1002 and needle 1004 are housed to be passed through, for example, a working channel of an endoscope to a target tissue. The stylet 1002 includes a shaft 1012 extending longitudinally from a proximal end to a distal end 1006. Similarly to the stylet 902, the distal end 1006 of the stylet 1002 includes a plunger element 1010 having a cross-sectional diameter larger than that of a remaining portion of the stylet 1002 and a lumen 1026 of the needle 1004 so that the plunger element 1010 extends distally of a distal end 1024 of the needle 1004 and is prevented from being drawn proximally thereinto. A cross-sectional area of the shaft 1012 may be only slightly smaller than a cross-sectional area of the lumen 1026 of the needle 1004 so that the stylet 1002 is longitudinally slidably within the needle 1004 between an open configuration in which the plunger element 1010 is spaced from the distal end 1024 of the needle 1004 and a closed configuration in which a proximal end 1014 of the plunger element 1010 is moved proximally toward the distal end 1024. The plunger element 1010 includes a tapered distal tip 1020 for piercing tissue into which it is inserted. The distal end 1024 of the needle 1004 may also include a sharpened edge for facilitating penetration of target tissue.

A distal portion of the stylet 1002 immediately distal of the plunger element 1010 includes a recess 1008 extending therealong for the collection of a tissue sample. Thus, in the open configuration, the recess 1008 is distal of the distal end 1024 of the needle 1004. As shown in FIG. 24, the recess 1008 includes a curved interior surface 1062 substantially corresponding to an exterior surface 1064 of the stylet 1002 to maximize a space within which the tissue sample may be received. Longitudinal edges 1066 of the recess 1008 may be sharpened so that, once the stylet 1002 has been inserted into the target tissue, the stylet 1002 may be rotated about a longitudinal axis thereof relative to the needle 1004 so that the longitudinal edges 1066 core the target tissue separating it from surrounding tissue to collect the tissue sample within the recess 1008. In other words, the sharpened longitudinal edges 1066 cut the target tissue as the stylet 1002 is rotated relative to the needle 1004. The longitudinal edges 1066, however, are not required to be sharpened.

In use, the device 1000 is inserted through the working channel of an endoscope or other insertion device in the closed configuration, with the stylet 1002 and the needle 1004 housed entirely within the sheath 1060. As indicated above, in this position, the plunger element 1010 does not extend distally of a distal end 1068 of the sheath 1060 and the sheath 1060 protects the endoscope from the sharpened distal end 1024 of the needle 1004 and the tapered tip 1020 of the plunger element 1010. Once the device 1000 has reached a desired position adjacent to or within target tissue, the needle 1004 and stylet 1002 are moved distally relative to the sheath 1060, in the closed configuration, to pass into the target tissue. In the target tissue, the stylet 1002 is moved distally relative to the needle 1004 from the closed configuration to the open configuration. The tissue is then compressed around the stylet 1002 such that rotation of the stylet 1002 relative to the needle 1004 cores a tissue sample from the surrounding target tissue, collecting the tissue sample within the recess 1008. As described above, sharpened longitudinal edges of the recess 1008 facilitate the cutting of the tissue sample from the surrounding tissue. Once the tissue sample has been cored, the stylet 1002 is drawn proximally relative to the needle 1004 from the open configuration to the closed configuration to hold the tissue sample in the recess 1008 via an interior surface 1070 of the needle 1004. Alternatively, the needle 1004 may be moved distally over the recess 1008, to move the device 1000 from the open configuration to the closed configuration. Once the device 1000 is in the closed configuration, the stylet 1002 and needle 1004 may be drawn proximally into the sheath 1060 to be removed from the patient's body.

Variations may be made in the structure and methodology of the present disclosure, without departing from the spirit and the scope of the disclosure. Thus, it is intended that the present disclosure cover the modifications and variations of this disclosure that may be contemplated by a person of skill in the art.

1-15. (canceled)

16. A device for collecting a tissue sample, comprising:
a needle extending longitudinally from a proximal end to a distal end and including a channel extending therethrough, the distal end including a pair of jaws, at least a first one of the jaws being movable relative to the other between an open configuration, in which the jaws are open to receive tissue therebetween, and a closed configuration in which the jaws are drawn toward one another to collect a tissue sample therein.

17. The device of claim 16, further comprising an oversheath extending longitudinally from a proximal end to a distal end and including a lumen extending therethrough, the lumen sized and shaped to slidably receive the needle therein.

18. The device of claim 16, wherein a first one of the jaws is pivotally coupled to the distal end of the needle while a second one of the jaws is fixed relative thereto.

19. The device of claim 18, wherein the first one of the jaws is coupled to the needle via a hinge and movable relative thereto via a control wire extending from a distal end connected to the first one of the jaws to a proximal end extending through the channel proximally of a proximal end of the needle.

20. The device of claim 18, wherein the first one of the jaws is coupled to the needle via a living hinge and is biased toward the open configuration.

21. The device of claim 16, wherein the jaws are formed via a longitudinal slot extending proximally from the distal end of the needle, each jaw of the pair of jaws coupled to a remaining portion of the needle via a living hinge and is biased toward the open configuration.

22. The device of claim 16, wherein a longitudinal edge of the jaws is sharpened to cut tissue as the jaws are moved to the closed configuration.

23. The device of claim 18, wherein the first one of the jaws includes a closed tip such that, when the jaws are in the closed configuration, a distal opening of the channel is at least partially closed.

24. The device of claim 16, wherein at least one of the jaws include a plurality of barbs extending inward into the channel from an interior surface of the jaws for gripping the tissue sample received therein.

25. The device of claim 16, wherein a first one of the jaws is positioned proximally of a tapered tip of the needle, a grind of the needle angled to guide tissue into the channel via the jaws when the jaws are in the open configuration.

26. A device for collecting a tissue sample, comprising:
a needle extending longitudinally from a proximal end to a distal end and including a lumen extending therethrough; and
a stylet including a shaft extending longitudinally from a proximal end to a distal end and a plunger element connected to the distal end of the shaft, the plunger element positioned distally of the distal end of the lumen such that the shaft extends proximally through the lumen of the needle, the stylet movable between an open configuration, in which the plunger is separated from the distal end of the needle, and a closed configuration, in which the plunger is moved proximally toward the distal end of the needle to guide a target tissue into the lumen of the needle.

27. The device of claim 26, wherein the plunger includes a tapered distal tip.

28. The device of claim 26, wherein a proximal surface of the plunger includes a recess for receiving the target tissue as it is guided into the lumen of the needle.

29. The device of claim 26, wherein an edge of the proximal surface is one of sharpened and includes barbs for cutting tissue.

30. The device of claim 26, wherein the shaft includes a recess extending along a distal portion thereof, the recess including sharpened longitudinal edges such that rotation of the stylet, in the open configuration, about a longitudinal axis relative to the needle cores a tissue sample from the target tissue and collects the tissue sample within the recess.

31. A method for collecting a tissue sample, comprising:
inserting a device to a target area within a patient body via a working channel of an endoscope, in a closed configuration in which jaws at a distal end of a needle are drawn toward one another;
inserting the needle into a target tissue while moving the jaws of the needle to an open configuration in which the jaws are separated from one another to receive a portion of the target tissue therebetween; and
moving the jaws to the closed configuration to collect a tissue sample within a portion of a channel of the needle defined between the jaws.

32. The method of claim 31, wherein moving the jaws of the needle between the open and closed configurations includes moving a control member distally and proximally, respectively, relative to the needle, the control member extending from a distal end connected to one of the jaws through the channel of the needle to a proximal end extending proximally of a proximal end of the needle.

33. The method of claim 31, wherein moving the jaws of the needle between the open and closed configurations includes sliding an oversheath distally relative to the jaws in the closed configuration and proximally of the jaws in the open configuration, an interior surface of the oversheath constraining the jaws in the closed configuration.

34. The method of claim 31, wherein longitudinal edges of the jaws cut the tissue sample from a surrounding target tissue as the jaws are moved from the open configuration to the closed configuration.

35. The method of claim 31, wherein barbs extending from an interior surface of the jaws into the portion of the channel defined thereby grip the tissue sample collected therein.
